SEQUENCE LISTING (10 sequences)

SEQ ID NO: 1 - DNA, 6967 bp, synthetic construct
  misc_feature 1..6967: note = vector
  source 1..6967: mol_type = other DNA
tcaaggttaggaacagagagacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgcgtcgcccgggtacccgtattcccaataaagcctcttgctgtttgcatcgggggtctttcatttggaggttccaccgagatttggagacccctgcccagggaccaccgacccccccgccgggaggtaagctggccagcggtcgtttcgtgtctgtctctgtctttgggcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagctcacttacaggcggccgcatggattttcaagtgcagattatcagcttcctgctaatcagtgcttcagtcataatgtccagaggacaaattgttctctcccagtctccagcaatcctgtctgcatctccaggggagaaggtcacaatgacttgcagggccagctcaagtgtaagttacatgcacttttaccaacagaagccaggatcctcccccaaaccctggatttatgccacatccaacctggcttctggagtccctgctcgcttcagtggcagtgggtctgggacctctttctctctcacaatcagcagagtggaggctgaagatgctgccacttattactgccagcagtggagtagtaacccacccacgttcggctcggggacaaagttggaaataaaatcaggtggaggagggtctggtggtggtggttctggcggaggaggctccatggcagtggttacaggggtcaattcagaggttcagctgcagcagtctggggcagagcttgtgaagccaggggcctcagtcaagttgtcctgcacagcttctggcttcaacattaaagacacctatatacactgggtgaagcagaggcctgaacagggcctggagtggattggaaggattgatcctgcgaatggtaatactaaatatgacccgaagttccagggcaaggccactataacaacagacacatccttcaacacagcctacctgcagctcagcagcctgacatctgaggacactgccgtctattactgtgctaaagtgggttacggccactggtacttcgatgtctggggcgcagggaccacggtcaccgtctcctcagtcgacaaggtgaacagcaccacaactaaacctgtcctgagaactcccagtcctgtgcacccaactggaacctcacagccacagcgaccagaggattgccgacctcgcgggagcgtgaagggaaccggactggacttcgcctgtgattctagtccaaaactcttttgggcactggtggtcgtggctggcgtgctcttttgctacggactcctggtcactgtggccctgtgcgtgatctggaccaactccaggagaaatagactcctgcaggtgaccacaatgaacatgacccctcggcgcccaggactgacacgcaagccataccagccttatgccccagccagggacttcgcagcatatagaccagcacacgcccgggctaagttcagcaggagcgccgagacagctgcaaacctccaggatcctaatcagctgtacaacgaactcaatctggggcgaagggaggaatatgacgtgctggagaagaaacgagcaagggatcccgaaatgggcggaaagcagcagagacggcgcaaccctcaggagggagtgtacaatgctctgcagaaggacaaaatggcagaggcctattccgaaattgggaccaagggtgaacgaaggagagggaaaggtcatgatggcctgtaccagggactgtccaccgctaccaaggatacctatgacgcactccacatgcagaccctcgcccccagatgagagaattcgagcatcttaccgccatttattcccatatttgttctgtttttcttgatttgggtatacatttaaatgttaataaaacaaaatggtggggcaatcatttacattttatgggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattccaccgtgaactttgtctcctggcatgcaaatcgtcaacttggcatgccaagaattaattcggatccaagcttaggcctgctcgctttcttgctgtcccatttctattaaaggttcctttgttccctaagtccaactactaaactgggggatattatgaagggccttgagcatctggattctgcctagcgctaagcttcctaacacgagccatagatagaataaaagattttatttagtctccagaaaaaggggggaatgaaagaccccacctgtaggtttggcaagctagcttaagtaagccattttgcaaggcatggaaaaatacataactgagaatagagaagttcagatcaaggttaggaacagagagacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccggggtacccgtattcccaataaagcctcttgctgtttgcatccgaatcgtggactcgctgatccttgggagggtctcctcagattgattgactgcccacctcgggggtctttcattctcgagagctttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgctggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagttactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaagccattttgcaaggcatggaaaaatacataactgagaatagagaagttcaga

SEQ ID NO: 2 - protein, 18 aa, synthetic construct
  REGION 1..18: note = vector
MDFQVQIISFLLISASVI

SEQ ID NO: 3 - protein, 110 aa, synthetic construct
  REGION 1..110: note = vector
MSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIK

SEQ ID NO: 4 - protein, 16 aa, synthetic construct
  REGION 1..16: note = vector
SGGGGSGGGGSGGGGS

SEQ ID NO: 5 - protein, 130 aa, synthetic construct
  REGION 1..130: note = vector
MAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVD

SEQ ID NO: 6 - protein, 47 aa, synthetic construct
  REGION 1..47: note = vector
KVNSTTTKPVLRTPSPVHPTGTSQPQRPEDCRPRGSVKGTGLDFACD

SEQ ID NO: 7 - protein, 76 aa, synthetic construct
  REGION 1..76: note = vector
SSPKLFWALVVVAGVLFCYGLLVTVALCVIWTNSRRNRLLQVTTMNMTPRRPGLTRKPYQPYAPARDFAAYRPAHA

SEQ ID NO: 8 - protein, 113 aa, synthetic construct
  REGION 1..113: note = vector
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 9 - DNA, 1533 bp, synthetic construct
  misc_feature 1..1533: note = vector
  source 1..1533: mol_type = other DNA
atggattttcaagtgcagattatcagcttcctgctaatcagtgcttcagtcataatgtccagaggacaaattgttctctcccagtctccagcaatcctgtctgcatctccaggggagaaggtcacaatgacttgcagggccagctcaagtgtaagttacatgcacttttaccaacagaagccaggatcctcccccaaaccctggatttatgccacatccaacctggcttctggagtccctgctcgcttcagtggcagtgggtctgggacctctttctctctcacaatcagcagagtggaggctgaagatgctgccacttattactgccagcagtggagtagtaacccacccacgttcggctcggggacaaagttggaaataaaatcaggtggaggagggtctggtggtggtggttctggcggaggaggctccatggcagtggttacaggggtcaattcagaggttcagctgcagcagtctggggcagagcttgtgaagccaggggcctcagtcaagttgtcctgcacagcttctggcttcaacattaaagacacctatatacactgggtgaagcagaggcctgaacagggcctggagtggattggaaggattgatcctgcgaatggtaatactaaatatgacccgaagttccagggcaaggccactataacaacagacacatccttcaacacagcctacctgcagctcagcagcctgacatctgaggacactgccgtctattactgtgctaaagtgggttacggccactggtacttcgatgtctggggcgcagggaccacggtcaccgtctcctcagtcgacaaggtgaacagcaccacaactaaacctgtcctgagaactcccagtcctgtgcacccaactggaacctcacagccacagcgaccagaggattgccgacctcgcgggagcgtgaagggaaccggactggacttcgcctgtgattctagtccaaaactcttttgggcactggtggtcgtggctggcgtgctcttttgctacggactcctggtcactgtggccctgtgcgtgatctggaccaactccaggagaaatagactcctgcaggtgaccacaatgaacatgacccctcggcgcccaggactgacacgcaagccataccagccttatgccccagccagggacttcgcagcatatagaccagcacacgcccgggctaagttcagcaggagcgccgagacagctgcaaacctccaggatcctaatcagctgtacaacgaactcaatctggggcgaagggaggaatatgacgtgctggagaagaaacgagcaagggatcccgaaatgggcggaaagcagcagagacggcgcaaccctcaggagggagtgtacaatgctctgcagaaggacaaaatggcagaggcctattccgaaattgggaccaagggtgaacgaaggagagggaaaggtcatgatggcctgtaccagggactgtccaccgctaccaaggatacctatgacgcactccacatgcagaccctcgcccccagatga

SEQ ID NO: 10 - DNA, 6967 bp, synthetic construct; identical to SEQ ID NO: 1
  misc_feature 1..6967: note = vector
  source 1..6967: mol_type = other DNA

(19) United States
(12) Patent Application Publication
(10) Pub. No.: US 2025/0215075 A1
(43) Pub. Date: Jul. 3, 2025

(54) Use of Car and Bite Technology Coupled with an SCFV from an Antibody Against Human Thymidine Kinase 1 to Specifically Target Tumors

(71) Applicants: Kim Leslie O'Neill, Provo, UT (US); Scott Weber, Lindon, UT (US)

(72) Inventors: Kim Leslie O'Neill, Provo, UT (US); Scott Weber, Lindon, UT (US)

(21) Appl. No.: 18/821,168

(22) Filed: Aug. 30, 2024

Related U.S. Application Data
(63) Continuation of application No. 17/366,708, filed on Jul. 2, 2021, now Pat. No. 12,076,378.
Application No. 17/366,708 is a continuation of application No. 16/122,564, filed on Sep. 5, 2018, now Pat. No. 11,052,138.
Application No. 16/122,564 is a continuation of application No. 15/161,045, filed on May 20, 2016, now Pat. No. 10,434,153.
(60) Provisional application No. 62/164,524, filed on May 20, 2015.
(60) Provisional application No. 62/204,935, filed on Aug. 13, 2015.

(51) Int. Cl. C07K 16/28; A61K 39/00; A61P 35/00; C07K 14/725; C07K 16/40; C07K 16/46

(52) U.S. Cl. CPC C07K 16/2803; A61K 39/0011; A61K 39/001162; A61P 35/00; C07K 14/7051; C07K 16/40; C07K 16/468; A61K 2039/505; A61K 2039/5156; A61K 2039/5158; C07K 2317/31; C07K 2317/34; C07K 2317/622; C07K 2319/02; C07K 2319/03

(57) ABSTRACT

Modified T-cells have paratopes against human TK1 epitopes, are made by producing monoclonal antibodies that are specific to TK1, creating chimeric antigen receptors (CARs) by fusion of the single-chain variable fragments (scFv) of the monoclonal antibodies to T-cell signalling domains, and transducing the CARs to the T-cells.

### CROSS REFERENCE TO RELATED APPLICATIONS

This application is a continuation of U.S. patent application Ser. No. 17/366,708, filed Jul. 2, 2021, now U.S. Pat. No. 12,076,378, issued on Sep. 3, 2024, which is a continuation of U.S. patent application Ser. No. 16/122,564, filed Sep. 5, 2018, now U.S. Pat. No. 11,052,138, issued on Jul. 6, 2021, which claims priority to U.S. patent application Ser. No. 15/161,045, filed May 20, 2016, now U.S. Pat. No. 10,434,153, issued on Oct. 8, 2019, which itself claims priority from U.S. Provisional Patent Application 62/164,524, filed May 20, 2015, and from U.S. Provisional Patent Application 62/204,935, filed Aug. 14, 2015, the entireties of which are hereby incorporated by reference.

### INCORPORATION BY REFERENCE

The ST.26 XML Sequence listing named “10616 US-ST26”, created on Aug. 30, 2024, and having a size of 49,152 bytes, is hereby incorporated herein by this reference in its entirety.

### BACKGROUND

Although efforts in cancer research have been ongoing for more than 20 years, cancer still accounts for more deaths than heart disease in persons younger than 85 years. Very little progress has been made in the past 30 years to decrease cancer mortality rates and incidence rates. In 2014, there will be an estimated 1,665,540 new cancer cases diagnosed and 585,720 cancer deaths in the US. Cancer remains the second most common cause of death in the US, accounting for nearly 1 of every 4 deaths. [1] It is accepted that while there has been some isolated progress with respect to a few types of cancers (breast, prostate, colon), overall, cancer researchers in the past 30 years have made very little headway against this devastating disease.

Although there has been very little progress, many novel chemotherapies that employ the use of specific antibodies are now showing promise in clinical trials. These new immunotherapies include the use of antibodies and immune cell therapies, coupled with cytokine administration, that are targeting specific tumor types and allowing significant progress in several cancer fields. Many of these chemotherapies target very specific subtypes of cancer, such as treatments with Herceptin (an antibody therapy) used in approximately 10% of breast cancers.

Antibodies against tumor associated epitopes, are proving useful in many tumor therapies but are limited to antigens presented on the cell surface of tumors. Several antibodies have been identified and exploited against multiple types of cancers using passive immunization. Notable examples include rituximab (anti-CD20 for B-cell lymphomas) and trastuzumab (anti-HER-2/neu for certain breast cancers). [6] Therapeutic antibodies have had success against tumors, eliciting both complement-mediated responses and antibody-dependent cellular cytotoxicity (ADCC). However, administration of an anti- cancer antibody as a monotherapy is rare, and these are often combined with more traditional chemotherapy. [4]

However, unless researchers are able to identify a cancer specific yet universal therapy to target all cancers, progress in the fight against cancer will also be limited. In the present study, we introduce the potential for one such novel immunotherapy: Thymidine Kinase 1 (TK1), a tumor biomarker known to be unregulated as an early event in virtually all types of major cancers.

Thymidine Kinase 1 (TK1) is a well-known nucleotide salvage pathway enzyme that has largely been studied in the context of its overexpression in tumors. Since TK was initially popularized by its expression in the serum of cancer patients (sTK), its diagnostic and prognostic potential has been studied extensively. For example, several studies have demonstrated that sTK1 in many different cancer patients is elevated in a stage-like manner with a higher level of TK1 indicating a more advanced tumor.

Other studies have investigated the prognostic potential of TK1. One such study demonstrates that the TK1 levels in primary breast tumors can be used to predict recurrence. Other exciting TK1 prognostic studies show significant reductions in sTK1 levels when patients respond to treatment while sTK1 levels continue to rise in patients who do not appear to respond to their treatment. It is also known that prior to recurrence, sTK1 levels begin to rise and in one study it was noted that in some cases, by measuring sTK1 levels recurrence could be predicted “1-6
months before the onset of clinical symptoms.” Several other studies confirm the rich potential of TK1 as a diagnostic and prognostic indicator of cancer [13].

Although the diagnostic and prognostic potential of TK1 has been well established, the therapeutic potential of TK1 remains veiled in comparison. While it is true that HSV-TK has been used in gene therapy and PET imaging utilizes TK to identify proliferating cancer cells, few, if any studies address the possibility of a TK1 immunotherapy. Perhaps this is primarily because TK1 is a known cytosolic protein. We have recently discovered that TK1 is expressed not only in cancer cells but also on the surface membrane of all cancer types and is therefore a very viable target for tumor immunotherapy.

T cells are capable of inducing potent anti-tumor responses, however, T cells that would most efficiently respond to peptide-MHC epitopes on the surface of tumors are often subjected to clonal tolerance or deletion, as many of these epitopes are very similar or identical to self-epitopes. T-cell therapies have involved genetic modification of T cells in vitro by introduction of TCRs against tumor-associated T-cell epitopes. This strategy has shown promise, but various challenges surrounding T-cell epitopes in general, as well as potential mispairing of introduced TCR with endogenous TCR, remain [3]. To harness the power of T cells in the fight against tumors, several methods have been designed that allow T cells to respond to traditional antibody epitopes.

Chimeric antigen receptors (CARs), consisting of extracellular antibody fragments directed against a tumor epitope fused to intracellular T-cell signaling domains, have been transduced into T cells, endowing them with a novel specificity toward a non-MHC restricted epitope [3]. Chimeric antigen receptors (CARs) are recombinant receptors that provide both surface antigen-binding and T-cell-activating functions. A number of CARs has been reported over the past decade, targeting an array of cell surface tumor antigens. Their biologic functions have dramatically changed following the introduction of tripartite receptors comprising a costimulatory domain, termed second-generation CARs.

These have recently shown clinical benefit in patients treated with CD19-targeted autologous T cells. CARs may be combined with costimulatory ligands, chimeric costimulatory receptors, or cytokines to further enhance T-cell potency, specificity, and safety. CARs represent a new class of drugs with exciting potential for cancer immunotherapy. [3]

Artificial T cell receptors (also known as chimeric T cell receptors, chimeric immunoreceptors, chimeric antigen receptors (CARs)) are engineered receptors, which graft an arbitrary specificity onto an immune effector cell. Typically, these receptors are used to graft the specificity of a monoclonal antibody onto a T cell; with transfer of their coding sequence facilitated by retroviral vectors.

Upon their expression in T lymphocytes, CARs direct potent, targeted immune responses that have recently shown encouraging clinical outcomes in a subset of patients with B-cell malignancies. This application brings together this new technology with our discovery that TK1 is expressed on the surface of cancer cells, by using a specifically built CAR that has an scFv from our anti human TK1 antibody to utilize the potential of CARs and the TK1 technology to attack tumor cells in vivo. Our recent discovery that TK1 is found on the surface of cancer cells and not normal cells allows the targeted application of CAR technology specific targeting TK1 surface expressing tumors.

The most common CAR formats currently being evaluated include a scFv targeting domain linked to a spacer, trans membrane domain, and intracellular domains from the T-cell receptor CD3s subunit and co-stimulatory domains, such as CD28, OX40 or 4-1BB.21CAR-based strategies continue to be pursued against a number of tumor-associated epitopes. [4]. Results from recent clinical trials demonstrate the effectiveness of CAR-transduced T cells targeted against the B cell epitope CD19 in achieving long-term remission from refractory chronic lymphocytic leukemia (CLL) when transferred as a monotherapy following lymphodepleting chemotherapy [5].

Referring to FIG. 1 is shown a TK1 specific CART cell that recognizes TK1 as a target on cancer cells. In the ligand binding domain ectodomain is shown a signal peptide and an antigen recognition domain is usually an scFv. A spacer region links the antigen binding domain to the transmembrane domain.

The transmembrane domain is a hydrophobic alpha helix that spans the membrane. Generally, the transmembrane domain from the most membrane proximal component of the endodomain is used.

After antigen recognition receptors cluster in the endodomain and a signal is transmitted to the cell. In an aspect of CARs, there is the intracellular domain from the CD3-zeta (CD3 ζ) chain, which is the primary transmitter of signals from endogenous T-cell receptors (TCRs). There may be added intracellular signaling domains from various costimulatory protein receptors, such as CD3-zeta and additional co-stimulatory signaling. ZAP-70 also is part of the T cell receptor, and plays a critical role in T-cell signaling.

Another strategy to target T cells to precise antibody epitopes takes advantage of a long-studied type of molecule called “bispecific antibody,” which links an anti-cancer antibody with an antibody recognizing CD3 subunits.

These have recently been termed BiTEs (bispecific T-cell engagers). A single-chain variable fragment (scFv) that binds a tumor epitope is linked to a second scFv that binds an invariant portion of the T-cell receptor complex, resulting in activation and targeting of effector T cells against the tumor epitope, regardless of the TCR-mediated specificity of the T cells. Evidence shows that these reagents are considerably more potent than antibodies against tumor cells alone. BiTEs have been constructed targeting more than ten tumor associated epitopes, including blinatumomab against CD19 (for B cell leukemias), and MT-110 against EpCAM (for various adenocarcinomas and cancer stem cells), both being currently evaluated in clinical trials. High response rates for relapse-free survival and elimination of minimal residual disease were found in refractory acute lymphoblastic leukemia (ALL) patients receiving blinatumomab in clinical trials [6].

Referring to FIG. 2, BiTEs are fusion proteins consisting of two single-chain variable fragments (scFvs) of different antibodies, or amino acid sequences from four different genes, on a single peptide chain. One of the scFvs binds to T cells via the CD3 receptor, and the other to a tumor cell via a tumor specific molecule,

Like other bispecific antibodies, and unlike ordinary monoclonal antibodies, BiTEs form a link between T cells and tumor cells. This causes T cells to exert cytotoxic activity on tumor cells by producing proteins like perforin and granzymes, independently of the presence of MHC I or co-stimulatory molecules. These proteins enter tumor cells and initiate the cell's apoptosis. This action mimics physiological processes observed during T cell attacks against tumor cells.

While both of these strategies have shown promising results, it is not yet clear under what conditions the CAR approach vs. the BiTE approach might be preferred. The optimal utilization of this knowledge would be in the production of a chimeric antigen receptor (CAR), or BiTEs utilizing the power of a monoclonal antibody produced against human TK1 coupled with the ability of T cells to destroy tumor cells. This is the basis of this application. Use your figure which is too general and make it more specific

FIG. 3 represents how engineered T-cells (by CARs ( ) can be used therapeutically by engineering cells from the patient's own body and infusing the T-cell back into the patient.

In BiTEs two single chain variable fragments are bound by a linker, one ScFv binds a tumor antigen and the other binds a tumor antigen, activating T cells and bringer closer them to the tumor cell, the antibody binds CD3 activating the T cell and the other just bind the tumor cell.

### SUMMARY

An aspect is technology that would allow the use of a CAR or BiTE produced with a scFv from a humanized or non-human mammal (such as mouse) monoclonal antibody to TK1, that could be used with appropriate genetic engineering to manipulate lymphocytes (possibly T cells but may include other immune cells) ultimately from a patient but not limited to such, to treat a disease such as cancer. That fact that TK1 is on the surface of cancer cells and not on the surface of any normal cell is a major part of the discovery, as this knowledge can be used to allow the T cells to be directed specifically to the tumor cells.

Another aspect lies in the fact that using our specifically generated antibodies to human TK1 we have discovered that TK1 is expressed on the surface of human cancer cells and not on the surface of normal cells and thereby can be used to target CARs and BiTEs to the tumors.

As aspect is CARs and BiTEs using the monoclonal antibodies, such as BYU 74 BYU 72 and CB 001 that we have developed that bind specifically to TK1 on the surface of cancer cells. Antibodies specific to human TK1 are disclosed in U.S. patent application Ser. No. 12/982,250, and U.S. Pat. Nos. 7,837,998, 7,311,906, and 5,698,409, which are hereby incorporated by reference, and F. Zhang, X. Shao, J.G. Robison, B.K. Murray, and K.L. O'Neill. Hybridoma. February 2001, 20(1): 25-34. doi:10.1089/027245701300060382.

The CARs technology and BiTEs technology used disclosed herein can be used to modify macrophages as well as T cells. This may be combined with macrophage tissue-specific promoters directed toward the cancer tissue to eliminate targeting of TK1 is the blood serum and direct to TK1 on cell surfaces.

### BRIEF DESCRIPTION OF DRAWINGS

FIG. 1A TK1 specific CART Cell recognizes a cancer cell using TK1 on the surface as a target. CAR T cells become activated upon recognition of the cancer cell inducing cell death by apoptosis and lysis.

FIG. 2 depicts a generalized prior-art BITEs.

FIG. 3 depicts a therapeutic method using engineered T cells.

FIG. 4 shows a portion of a CARs transduced T-cell.

FIG. 5 is a construct of the TK1 CART cell vector. Retroviral mediated gene transfer. 293GPG human retroviral packaging cells are transfected with the vector of interest, which is packaged transiently in vesicular stomatitis virus (VSV) G pseudotyped particles. These particles are used to deliver the vector to PG13 cells, which achieve stable packaging of GALV pseudotyped particles that are suitable for infection of human T-cells.

FIG. 6 shows a method for transduction illustrating the retroviral mediated gene transfer.

FIGS. 7A-7D illustrate the sequence of the TK1 CAR T cell DNA vector (SEQ ID NO: 1). FIGS. 7A and 7B are an annotated TK1 CAR T Cell vector nucleotide map. FIGS. 7D and 7C are a FASTA version of the TK1 CAR T Cell vector nucleotide map.

FIG. 8 shows the sequence of the TK1 T cell CAR protein as FIG. 5. Therein depicted are the amino acid sequence of the signal peptide (SEQ ID NO:2): the CB1 light chain (SEQ ID NO:3): the glycine-serine linker (SEQ ID NO:4) the CB1 heavy chain (SEQ ID NO:5): the CD8a hinge (SEQ ID NO:6); the CD28 costimulatory domain (SEQ ID NO:7): and the CD3 zeta costimulatory domain (SEQ ID NO:8).

FIG. 9 TK1 CAR T cell Nucleotide (SEQ ID NO:1) and protein sequence (SEQ ID NOs:2-8 linked in order) alignment.

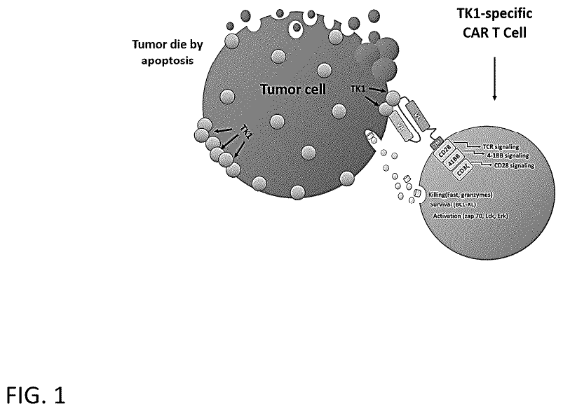

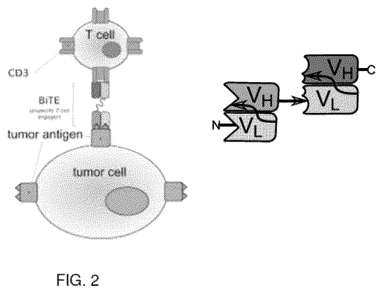

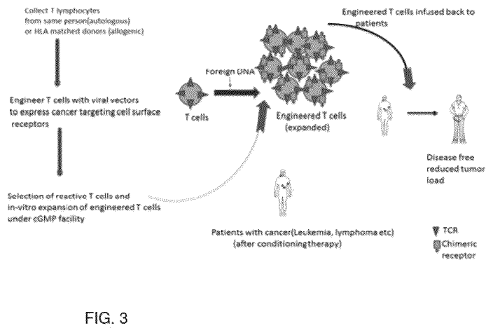

### DETAILED DESCRIPTION

### Example

This is a specific example of how CAR transduced T-cells can be made.

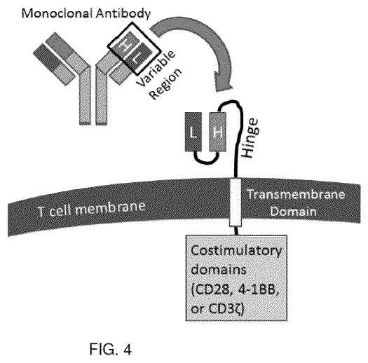

Reference is made to FIG. 4. A CARs transduced T-cells comprise single-chain variable fragments (scFv) from the variable region of a monoclonal antibody. In this example the monoclonal antibody is specific to human thymidine kinase 1 (TK1)

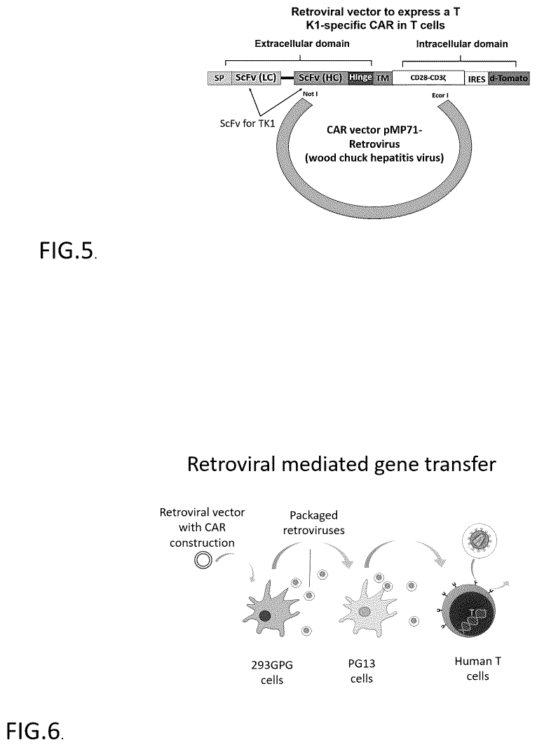

FIG. 5 is a schematic of a construct the signal peptide to which a chimeric antigen receptor will be added, which protein will be tranducted into the T-cells. It comprises an ectodomain signalling peptide based upon CB1 chains (k light chain attached to the scFv) and y heavy chain), a hinge portion based upon CDS, and an endodomain with costimulatory domains based upon CD28, CD3 zeta costimulatory protein receptors.

FIG. 6 illustrates a method for introducing any CAR protein by transduction into a T-cell and can be used in the present process. Chimeric antigen receptors (CARs) are genetically delivered fusion molecules that elicit T-cell activation upon binding of a native cell surface molecule. These molecules can be used to generate a large number of memory and effector T-cells that are capable of recognizing and attacking tumor cells. Most commonly, stable CAR expression is achieved in T-cells using retroviral vectors. In the method shown in FIG. 6, retroviral vectors are packaged in a two-step procedure. First, H29D human retroviral packaging cells (a derivative of 293 cells) are transfected with the vector of interest, which is packaged transiently in vesicular stomatitis virus (VSV) G pseudotyped particles. These particles are used to deliver the vector to PG13 cells, which achieve stable packaging of gibbon ape leukemia virus (GALV)-pseudotyped particles that are suitable for infection of human T-cells.

The key advantage of the method reported here is that it robustly generates polyclonal PG13 cells that are 100% positive for the vector of interest. This means that efficient gene transfer may be repeatedly achieved without the need to clone individual PG13 cells for experimental pre-clinical testing. To achieve T-cell transduction, cells must first be activated using a non-specific mitogen.

Phytohemagglutinin (PHA) provides an economic and robust stimulus to achieve this. After 48-72 h, activated T-cells and virus-conditioned medium are mixed in RetroNectin-coated plasticware, which enhances transduction efficiency.

Transduced cells are analyzed for gene transfer efficiency by flow cytometry 48 h following transduction and may then be tested in several assays to evaluate CAR function, including target-dependent cytotoxicity, cytokine production and proliferation. (See Parente-Pereira AC, Wilkie S, van der Stegen SJC, Davies OM, Maher J. Use of retroviral-mediated gene transfer to deliver and test function of chimeric antigen receptors in human T-cells. J Biol Methods 2014;1 (2):e7. doi: 10.14440/jbm.2014.30)

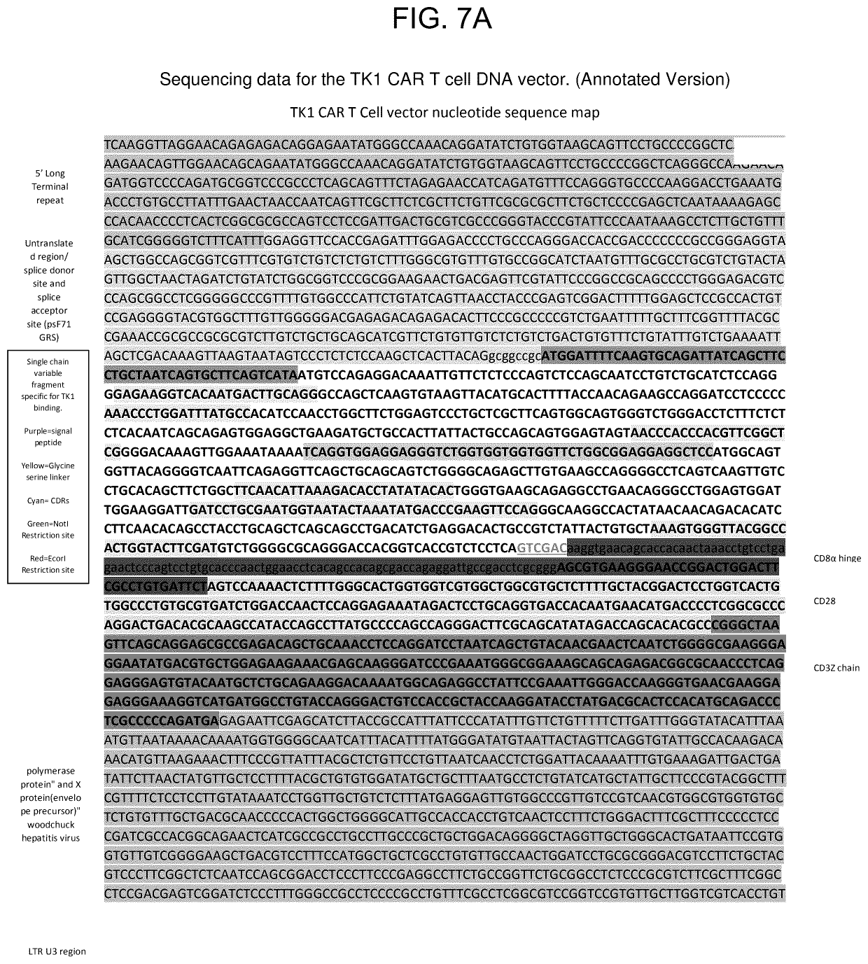

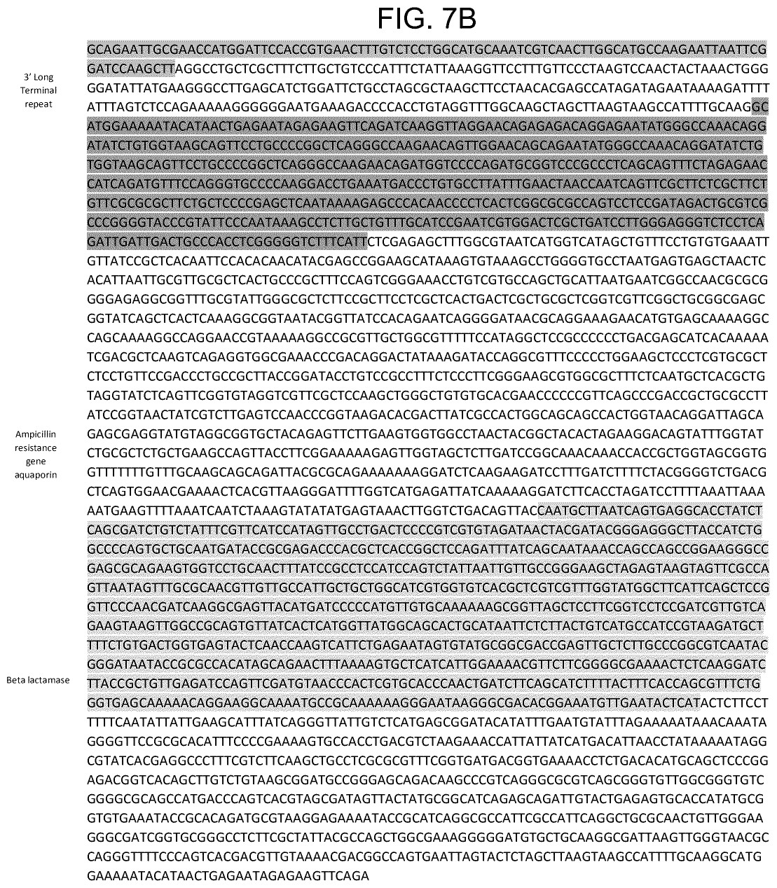

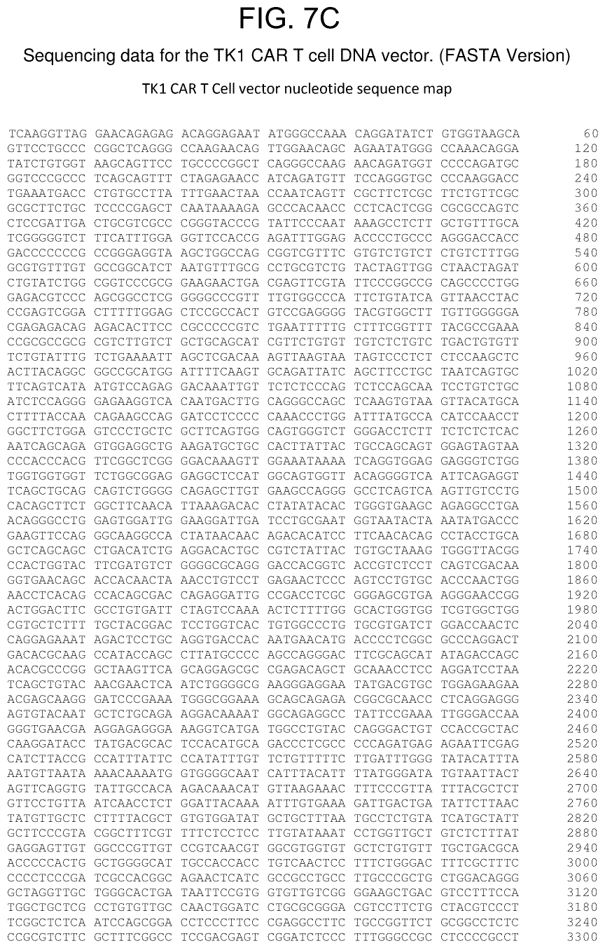

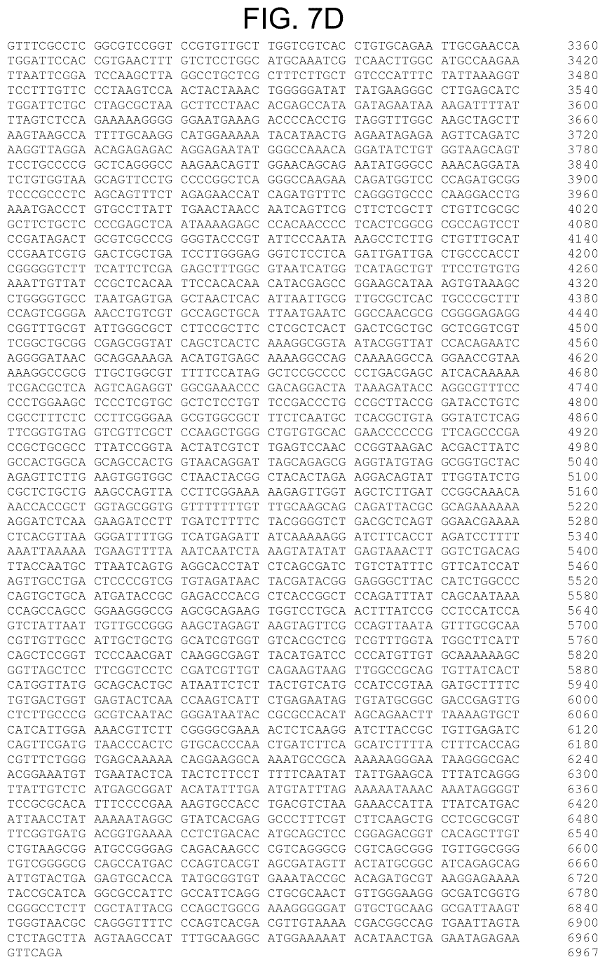

FIGS. 7A-7D illustrate the sequence of the DNA of the TK1 CART cell vector

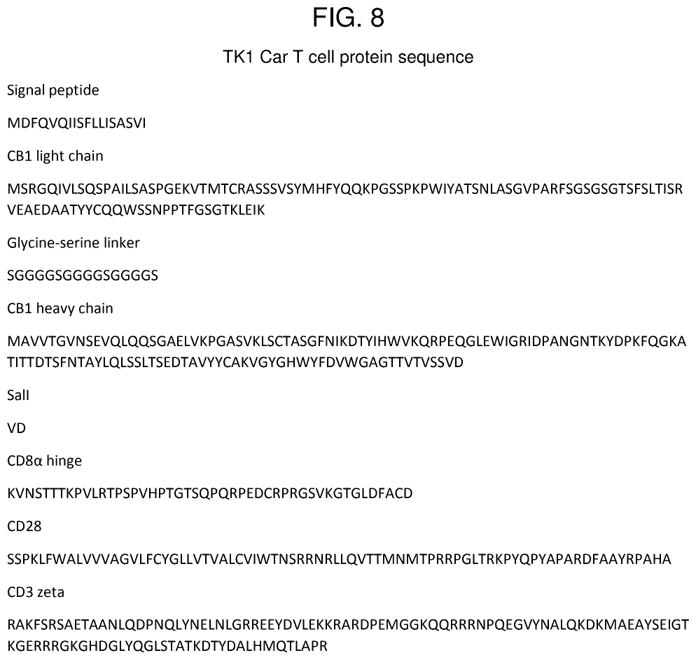

FIG. 8 shows the protein sequence of the TK1 CART cell protein

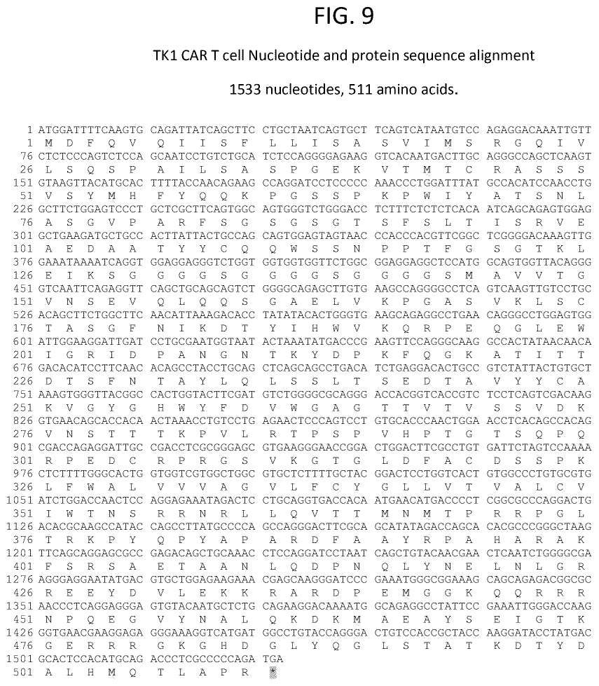

FIG. 9 shows the TK1 CART cell Nucleotide and protein sequence alignment

While this invention has been described with reference to certain specific embodiments and examples, it will be recognized by those skilled in the art that many variations are possible without departing from the scope and spirit of this invention, and that the invention, as described by the claims, is intended to cover all changes and modifications of the invention which do not depart from the spirit of the invention.

### REFERENCES

1. American Cancer Society, Cancer Facts and Figures. 2015.
2. Schreiber H. Tumor-specific immune responses. SeminImmunol 2008; 20:265-6; PMID:18977672; http://dx.doi.org/10.1016/j.smim.2008.10.001.
3. Stone, J.D. Aggen, D.H., Scheitinger, A, Schreiber, H, and Kranz, D.M. 2012 A sensitivity scale for targeting T cells with Chimeric Antigen Receptors (CARs) and Bispecific T-cell engagers (BiTEs) Onclommunology 1:6, 863-873
4. Schreiber H. Cancer Immunology. Philadelphia, PA: Lippincott-Williams & Wilkins 2012.
5. Karyampudi L, Knutson KL. Antibodies in cancer immunotherapy. Cancer Biomark 2010; 6:291-305; PMID:20938089.
6. Grillo-L.pez AJ, White CA, Varns C, Shen D, Wei A, McClure A, et al. Overview of the clinical development of rituximab: first monoclonal antibody approved for the treatment of lymphoma. Semin Oneal 1999; 26:66-73; PMID:10561020.
7. Goldenberg MM. Trastuzumab, a recombinant DNA derived humanized monoclonal antibody, a novel agent for the treatment of metastatic breast cancer. Clin Ther 1999; 21:309-18; PMID:10211534; http://dx.doi. org/10.1016/S0149-2918(00)88288-0.
8. Seliger B, Cabrera T, Garrido F, Ferrone S. HLA class I antigen abnormalities and immune escape by malignant cells. Semin Cancer Biol 2002; 12:3-13; PMID: 11926409; http://dx.doi.org/10.1006/scbi.2001.0404.
9. Garrido F, Cabrera T, Concha A, Glew S, Ruiz-Cabello F, Stern PL. Natural history of HLA expression during tumour development. Immunol Today 1993; 14:491 9; PMID:8274189; http://dx.doi.org/10.1016/0167-5699(93)90264-L.
10. Meidenbauer N, Zippelius A, Pittet MJ, Laumer M, Vogl S, Heymann J, et al. High frequency of functionally active Melan-a-specific T cells in a patient with progressive immunoproteasome-deficient melanoma. Cancer Res 2004; 64:6319-26; PMID:15342421;http://dx.doi.org/10.1158/0008-5472.CAN-04-1341.
11. Yu Z, Theoret MR, Touloukian CE, Surman DR, Garman SC, Feigenbaum L, et al. Poor immunogenicity of a self/tumor antigen derives from peptide-MHCI instability and is independent of tolerance. J Clin Invest 2004; 114:551-9; PMID:15314692.
12. Alegre. M, Robison, R.A. and O′Neill, K.L. Thymidine Kinase 1: A Universal Marker for Cancer. 2013 Cancer and Clinical Oncology 2013 vol 2: No 1; p 159-167.
13. O'Neill, K. L., Buckwalter, M. R., & Murray, B. K. (2001). Thymidine kinase: diagnostic and prognostic potential. Expert Rev Mal Diagn, 1 (4), 428-433. http://dx.doi.org/10.1586/14737159.1.4.428

1. A chimeric antigen receptor (CAR) comprising a targeting domain operatively linked to a signaling domain that polarizes a macrophage to an M1 macrophage;
wherein the CAR further comprises a linker and/or a transmembrane domain separating the targeting domain and the signaling domain.
wherein at least one of the targeting domain, the linker, or transmembrane domain is heterologous to the signaling domain.

2. The CAR of claim 1, wherein the binding domain is a single-chain variable fragment (scFv).

3. The CAR of claim 2, wherein the scFv is specific for a human antigen.

4. The CAR of claim 1, wherein the signaling domain that polarizes a macrophage to an M1 macrophage is a human signaling domain that polarizes a macrophage to an M1 macrophage.

5. The CAR of claim 1, wherein the signaling domain that polarizes a macrophage to an M1 macrophage is a TLR4 domain.

6. A cell comprising the CAR of claim 1.

7. The cell of claim 6, wherein the cell is a monocyte or a macrophage.

8. The cell of claim 7, wherein the monocyte is a human monocyte or wherein the macrophage is a human macrophage.

9. The CAR of claim 1, wherein the transmembrane domain is a hydrophobic alpha helix.

10. A chimeric antigen receptor (CAR) comprising a targeting domain operatively linked to a signaling domain that polarizes a macrophage to an M1 macrophage;
wherein the CAR further comprises a costimulatory domain.
wherein at least two of the targeting domain, the costimulatory domain and, the signaling domain are heterologous to each other.

11. The CAR of claim 10, wherein the binding domain is a single-chain variable fragment (scFv).

12. The CAR of claim 11, wherein the scFv is specific for a human antigen.

13. The CAR of claim 10, wherein the signaling domain that polarizes a macrophage to an M1 macrophage is a human signaling domain that polarizes a macrophage to an M1 macrophage.

14. The CAR of claim 10, wherein the signaling domain that polarizes a macrophage to an M1 macrophage is a TLR4 domain.

15. A cell comprising the CAR of claim 10.

16. The cell of claim 15, wherein the cell is a monocyte or a macrophage.

17. The cell of claim 16, wherein the monocyte is a human monocyte or wherein the macrophage is a human macrophage.

18. The CAR of claim 10, wherein the costimulatory domain is selected from the group consisting of CD28, OX40, 4-1BB.21, and CD3-zeta costimulatory domains.
